(19) United States
(12) Patent Application Publication
(10) Pub. No.: US 2018/0344577 A1
(43) Pub. Date: Dec. 6, 2018

(54) Double Lumen Integrated Enteral Feeding Assembly and a Method for Use Thereof

(71) Applicant: Angelo Coppola, Little Rock, AR (US)

(72) Inventor: Angelo Coppola, Little Rock, AR (US)

(21) Appl. No.: 15/608,606

(22) Filed: May 30, 2017

(51) Int. Cl. A61J 15/00

(52) U.S. Cl. CPC A61J 15/0069; A61J 15/0011; A61M 25/0032; A61J 15/0003; A61J 15/0073

(57) ABSTRACT

A dual-tube feeding tube assembly for single-procedure placement inside the body of a patient, the assembly having a tubing unit including a gastric tube and jejunal tube adjacent one another such that the proximal end of the tubing unit is positioned outside the patient's body and the distal end is positioned inside the patient's body. A pointed tip attachment piece on the proximal end of the tubing unit allows the tubing unit to be pulled orally through the patient's esophagus and stomach until the pointed tip attachment piece extends through an abdominal incision. A set of bumpers on opposite sides of the abdomen wall maintain the position of the tubing unit such that the distal end remains inside the body and the proximal end remains outside the body so that medicine, food, and other substances can be supplied to the patient through the tubing unit.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

Enteral feeding is the preferred method for administering nutrients and medication to individuals unable to intake these materials orally. Traditionally enteral feeding has been done with gastric feeding tubes that administer nutrients directly into the stomach. Because the stomach is directly connected to the esophagus, however, aspiration is possible and can pose a serious risk. Gastro-jejunal feeding tube systems, which have tubes in both the stomach and jejunum, significantly decrease this risk because nutrients can be delivered far from the esophagus through the jejunal tube and the stomach can be vented or drained through the gastric tube. However, the placement of each of these tubes requires a separate procedure (typically done under fluoroscopy) to allow for the stomach to fuse with the abdominal wall after the gastric tube has been placed and before the jejunal tube can be placed. Having multiple procedures requires a significant amount of both the patient's and surgeon's time. Furthermore, these procedures can be expensive for the patient and can increase the patient's risk for experiencing surgical complications. Therefore, there is a need in the enteral feeding field to create a new gastro-jejunal feeding tube that can be placed in a single procedure and an associated method for placing it.

### BRIEF SUMMARY OF THE INVENTION

This invention relates generally to the enteral feeding field, and more specifically to a new and useful gastro-jejunal feeding tube assembly for enteral feeding and a method for placing the feeding tube assembly endoscopically. It is an object of the present invention to provide a single integrated device useful for enteral feeding that decreases the risk of aspiration and decreases the amount of procedures necessary to place the tubes in the patient's body.

### BRIEF DESCRIPTION OF THE DRAWINGS

FIG. 1 depicts one embodiment of the feeding tube assembly for enteral feeding of the present invention placed inside a patient's body.

FIG. 2 is a side view of one embodiment of an enteral feeding tube assembly of the present invention showing the assembly outside of a patient's body.

FIGS. 3A-3D are side views of various alternative embodiments for the pointed tip attachment piece of the enteral feeding tube assembly of the present invention.

FIGS. 4A-4F are cross-sectional views of various alternative embodiments of the tubing unit and lumen arrangement of the feeding tube assembly of the present invention.

FIGS. 5A-5B are side views of various alternative embodiments of the longitudinal arrangement of the lumens of the feeding tube assembly of the present invention.

FIGS. 6A-6D are side views of various alternative embodiments of the feeding tube assembly having a split proximal end.

FIGS. 7A-7C are cross-sectional views of various alternative embodiments for the bumpers of the feeding tube assembly of the present invention.

FIGS. 8A-8B are side views of various alternative embodiments for the weighted tip attachment piece of the feeding tube assembly of the present invention.

FIGS. 9A-9D are perspective views of additional alternative embodiments for the weighted tip attachment piece of the feeding tube assembly of the present invention.

FIG. 10 depicts one embodiment of the feeding tube assembly for enteral feeding placed inside a patient's body with the feeding tube assembly having a split proximal end.

FIG. 11 is a flowchart outlining the steps of the preferred method for endoscopically placing the gastro-jejunal enteral feeding tube assembly of the present invention inside a patient's body.

### DETAILED DESCRIPTION OF THE INVENTION

The following description of the preferred embodiments of the invention is not intended to limit the invention to these preferred embodiments, but rather to enable any person skilled in the art of enteral feeding to make and use this invention.

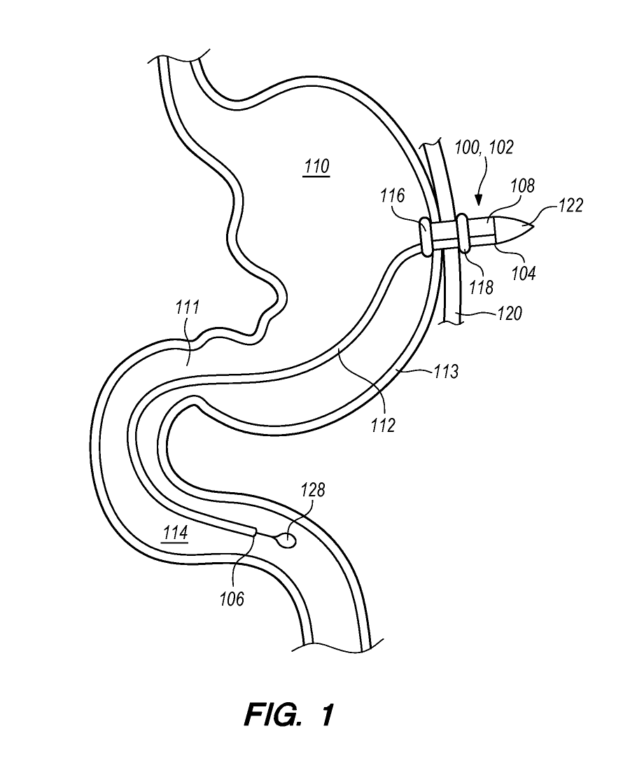

With reference to FIGS. 1-11, a device for enteral feeding according to the present invention may be described. The device generally comprises two interconnected tubes that extend in a side-by-side orientation with respect to each other. One tube is configured to function as a jenjunal tube and the other is configured to function as a gastric tube 108. Each tube comprises a hollow passageway, or lumen, through which food, medicine, or other substances may be delivered to the appropriate part of the patient's body. Alternatively, a single tube may be used, with the tube having two different lumens, one for delivery of substances to the stomach and one for delivery of substances to the jejunum. Thus, while the proximal end of both the gastric lumen and jejunal lumen align, the distal end of the jejunal lumen extends to the jejunum while the distal end of the gastric lumen terminates in the stomach. In addition, while the term lumen is generally used to refer to the hollow passageway portion of a tube, the terms “tube” and “lumen” may be used interchangeably herein for purposes of describing the delivery routes to different parts of the body. Thus, even if the term “lumen” is used to describe separate feeding delivery paths, it is understood that multiple tubes may serve the same purpose. Furthermore, the term “tubing unit” may be used herein to describe these tubes or lumens in combination, meaning that “tubing unit” refers to both the gastric tube/lumen and the jejunum tube/lumen. Finally, the term “dual-lumen length” hereby refers to the longitudinal segment of the tubing unit in which the gastric tube/lumen and jejunal tube/lumen are side-by-side (therefore the term “dual-lumen length” does not include the portion of the jejunal tube/lumen that extends beyond the distal end of the gastric tube/lumen).

With reference to FIG. 1, the preferred embodiment of the enteral feeding tube assembly 100 according to the present invention may be described. In one embodiment, the feeding tube assembly 100 comprises: a tubing unit 102 having a proximal end 104 located outside the patient's body and a distal end 106 located within the patient's body; a gastric lumen 108 starting at the proximal end 104 of tubing unit 102 and extending into the stomach 110; a jejunal lumen 112 starting at the proximal end 104 of tubing unit 102 and extending into the jejunum 114 (note, the proximal end 104 of the tubing unit 102 refers to both the proximal end of the gastric lumen 108 and jejunal lumen 112, while the distal end 106 of the tubing unit 102 is refers only to the distal end of the jejunal lumen 112 because the gastric lumen 108 is shorter than the jejunal lumen 112); a distal bumper 116 that extends perpendicularly from the surface of the tubing unit 102 at the most distal portion of the gastric lumen 108 (located inside the patient's body) configured to maintain appropriate placement of the tubing unit 102; a proximal bumper 118 that extends perpendicularly from the surface of the tubing unit 102 at the location just exterior to the abdominal wall 120 configured to maintain appropriate placement of the tubing unit 102; and a pointed tip attachment piece 122 connected to the proximal end 104 of the tubing unit 102.

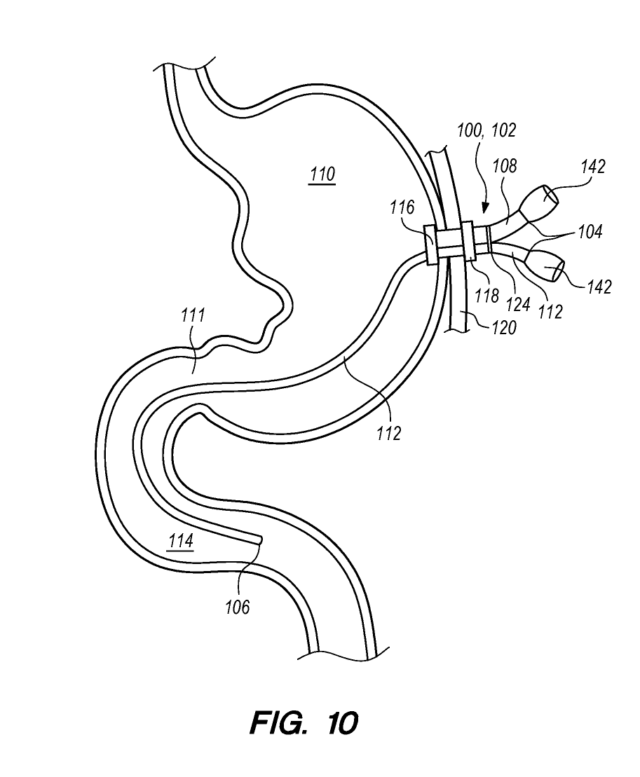

As shown in FIG. 10, alternative embodiments of the feeding tube assembly 100 further comprise the tubing unit 102 being split between the lumens 108, 112 for a prescribed length at its proximal end 104 and a retention band 124 or series of retention bands that circumscribe the tubing unit 102 to prevent further distal splitting of the tubing. As shown in FIG. 3D, in one embodiment an appendage 126 is connected to the pointed tip attachment piece 122. The appendage 126 is configured to be attached to a wire used in placement of the feeding tube assembly 100 in the patient's body (the method of which is described more fully below). Furthermore, as shown FIG. 1, the feeding tube assembly 100 may further comprise a weighted tip 128 attached to the most distal end 106 of the tubing unit 102 (and therefore the distal end of the jejunal lumen 112) configured to assist and maintain placement of the jejunal lumen 112 within the jejunum 114.

The tubing unit 102 functions to facilitate the passage of materials between the outside of the body and within two different regions of the gastrointestinal system: the stomach 110 and the jejunum 114. The tubing unit 102 defines two distinct tubes or lumens, a gastric lumen 108 that terminates in the stomach 110 and a jejunal lumen 112 that terminates in the jejunum 114. As noted above, the jejunal lumen 112 is longer than the gastric lumen 108 (by virtue of extending through the stomach to the small intestine). In one embodiment, for example, the jejunal lumen 112 is longer than the gastric lumen 108 by approximately 40 cm, with the jejunal lumen having a length of approximately 75 cm and the gastric lumen having a length of approximately 35 cm. These lengths, however, may be modified in order to adapt to the size of a particular patient's gastrointestinal area. In one embodiment, the tubing unit 102 comprises a sheath that envelops the tubing unit 102 at the proximal end 104 of the tubing unit 102. The sheath functions to provide an additional means of connecting the gastric tube 108 and jejunal tube 112. The sheath may serve as a means of bundling two separate tubes (gastric tube 108 and jejunal tube 112) or alternatively may envelop a single tube that comprises two separate lumens. In one embodiment, the jejunal tube and gastric tube are individual tubes bound by the sheath but are not otherwise connected. Alternatively, the jejunal tube and gastric tube are joined by an adhesive material in addition to being bundled by a sheath. In yet another embodiment, the tubing unit is cast as a single tube with two lumens, with the sheath enveloping the single tube. In any embodiment, the sheath serves as an additional means for keeping the tubing unit in tact as a single unit.

The inclusion of both lumens 108, 112 in a single tubing unit 102 allows for the entire feeding tube assembly 100 to be placed in a single endoscopic procedure (the method of which is described below), because the lumens 108, 112 are constrained to move together during placement. In the preferred embodiment, the tubing unit 102 is made of a flexible polymer such as silicone and manufactured as a single piece through extrusion. Alternatively, the tubing unit 102 can be manufactured through dip-coating, casting, 3D-printing, or other methods that would be well-known to a person of ordinary skill in the art. Furthermore, the tubing unit 102 can be formed from tubes manufactured separately that are then combined using adhesives, shrink wrap, bands, ties, sutures, or other means for secure attachment along the entire dual-lumen length of the tubing unit 102 such that the tubes 108, 112 are constrained to move together when placed using a pull-through endoscopic procedure. The tubing unit 102 can alternatively be made from such materials as polyurethanes, rubbers, or any other flexible materials that are non-toxic to the human body. Preferably, a friction reduction coating, such as Teflon, is applied to the surface of the tubing unit 102 to reduce friction as it is placed. Alternatively, other forms of surface coating can be used, or the tubing unit 102 can be non-coated.

In one embodiment, the proximal end 104 of the tubing unit 102 is configured to attach the proximal end of the jejunal tube 112 and gastric tube 108 to ports after placement of the feeding tube assembly 100 in the patient's body. These ports allow for the introduction of food and/or medicine into the patient's body through the appropriate tube 108, 112. Preferably, the dual-lumen length of tubing unit 102 (that is, the portion of the tubing unit 102 that is external to the abdominal wall 120) is approximately 20 cm in length. A proximal bumper 118 (discussed below) is preferably placed on this portion of the tubing unit 102 at approximately 3 to 5 cm from the abdominal wall 120 in order to keep the tubing unit 102 in the appropriate place after placement into the patient's body. Furthermore, a retention band 124 (discussed below) or multiple retention bands 124 may also be placed on the portion of the tubing unit 102 external to the patient's body. This retention band 124 will allow the portion of the tubing unit 102 external to the body to be trimmed down, as the biggest wear point is where the ports attach to tube feeds, while also preventing splitting of the unit beyond that purposefully split by the medical professional. The ports are preferably attached to the ending of the tubes after the tubing has been cut to the desired length. Additionally, tubing unit 102 preferably has an oval-shaped cross section 130 along its dual-lumen length with the lumens 108, 112 located side-by-side, as shown in FIG. 4A.

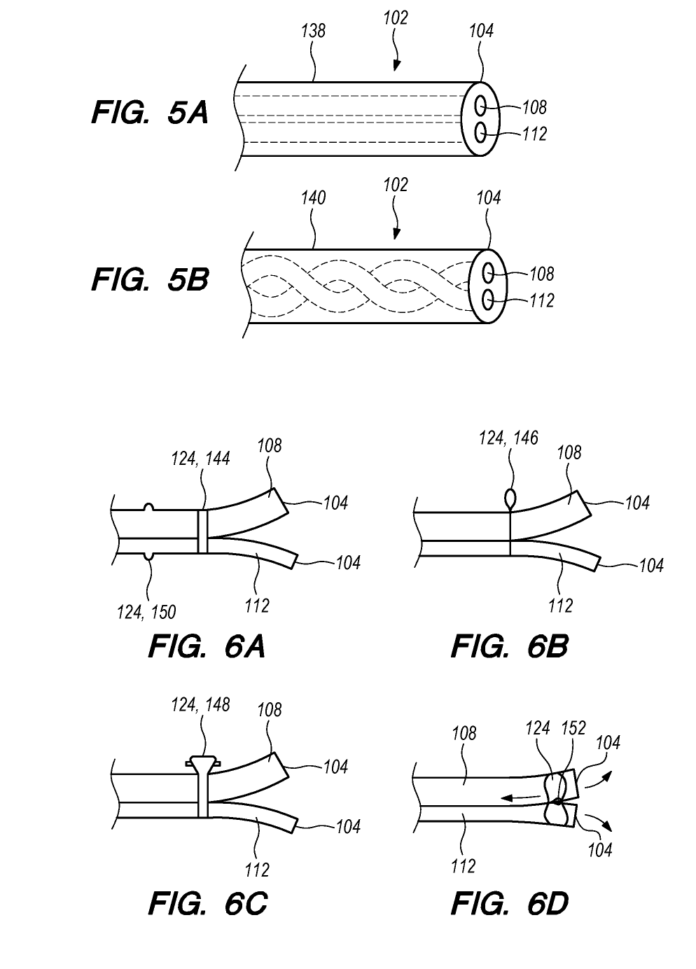

Alternatively, the tubing unit 102 cross section can have a figure-eight shape 132 (FIGS. 4C-4D), an asymmetric shape 134 (FIG. 4B), or any other geometric shape configured to keep the lumens 108, 112 separate from each other. Furthermore, the two lumens 108, 112 can be circumferentially arranged 136 (FIGS. 4E-4F). Preferably, the largest distance between any two points on the cross section of the tubing unit 102 will be within the range of 8-40 French. French gauge is commonly used to measure the size of tubing, with the French size being three times the size in millimeters. Thus, the preferred largest distance between any two points on the cross section of the tubing unit 102 will be within the range of 2.67 mm and 13.33 mm. The cross section can, however, have any dimensions that allow the tubing unit 102 to non-obstructively pass through the patient's esophagus. Preferably, the two lumens 108, 112 run parallel 138 to each other along the entire dual-lumen length of the tubing unit 102, as shown in FIG. 5A. Alternatively, the two lumens 108, 112 can curve along this length, such as in a helical arrangement 140, as shown in FIG. 5B. This modification of the arrangement of the two lumens 108, 112 may be made to adjust the profile of the device. For example, the helical arrangement 140 may provide a circular outer cross-section with a lower total profile than does the side-by-side arrangement 138, which in some embodiments would require an oblong shaped tubing (by cross section) or would require a larger outside diameter tubing. Such tubing sizes may impair healing time of the incision in the abdominal wall and therefore the tubing is preferably designed to reduce tubing size. Thus, the preferred embodiment implements the helical design 140 in order to reduce the outer diameter size requirements for the tubing unit 102; however, any arrangement may be used so long as the tubing unit 102 is constrained to move together during placement.

The gastric lumen 108 functions to transport materials between the outside of the body and the inside of the stomach 110. In many cases, it is desirable to place medicine in the stomach 110 so that it has sufficient time to be absorbed; the gastric lumen 108 allows for this administration. The gastric lumen 108 also provides a passage for gastric contents to exit the body in the case of aspiration or for venting. In the preferred embodiment, the gastric lumen 108 has a circular cross section with a diameter in the range of 8-16 French (2.67 mm to 5.33 mm). Preferably, the diameter (or greatest distance between points on the circumference, in the case of a noncircular lumen cross section) of the gastric lumen 108 is the same size or larger than the diameter (or greatest distance between points on the circumference, in the case of a noncircular lumen cross section) of the jejunal lumen 112, because the gastric lumen 108 is preferred for medicine administration, which can require larger passageways than food administration. Alternatively, the gastric lumen 108 cross section can be noncircular. Furthermore, the gastric lumen's 108 cross section can vary along its length. In some embodiments, the gastric lumen 108 can be coated with a friction reduction coating, such as Teflon, to allow for materials to more easily pass through it.

The jejunal lumen 112 functions to transport materials between the outside of the body and past the pylorus 111 into the jejunum 114. In most cases, it is preferable to place food in the jejunum 114 as opposed to directly in the stomach 110, because the food is less likely to be aspirated. The jejunal lumen 112 could also provide a passageway for the administration of medicine. In the preferred embodiment, the jejunal lumen 112 has a circular cross section with a diameter in the range of 4-12 French (1.33 mm to 4 mm). Preferably, the diameter (or greatest distance between points on the circumference, in the case of a noncircular lumen cross section) of the jejunal lumen 112 is the same size or smaller than the diameter (or greatest distance between points on the circumference, in the case of a noncircular lumen cross section) of the gastric lumen 108, to minimize the overall size of the tubing unit 102. Alternatively, the cross section of the jejunal lumen 112 can be noncircular. Furthermore, the jejunal lumen's 112 cross section can vary along its length. In some embodiments, the jejunal lumen 112 can be coated with a friction reduction coating, such as Teflon, to allow for materials to more easily pass through it.

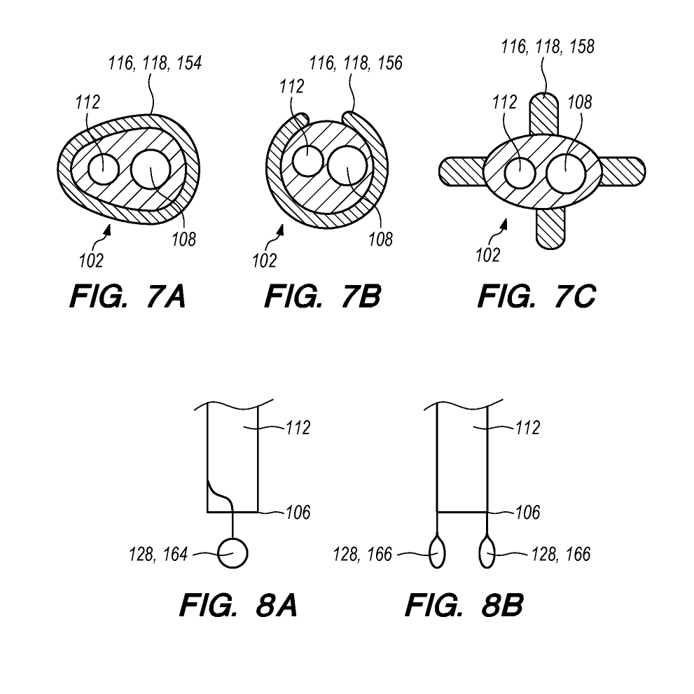

The distal bumper 116 is located on the tubing unit 102 at the distal end of the gastric lumen 108. It functions to secure the tubing unit 102 within the body and is configured to prevent the gastric outlet (the distal end of the gastric lumen 108) from exiting the stomach 110. In the preferred embodiment, the distal bumper 116 extends 1-5 mm perpendicularly from the outer surface of the tubing unit 102. Alternatively, any dimension that prevents the gastric outlet from exiting in the stomach 110 while allowing the tubing unit 102 to pass through the esophagus without causing obstruction or damaging internal tissue is appropriate. Various embodiments of the distal bumper 116 are shown in FIGS. 7A-7C. In the preferred embodiment, the distal bumper 116 is a ring composed of the same flexible material as the tubing unit 102, so that it minimizes the chance of obstruction or harm to any internal tissue as it is being placed. Alternatively, the distal bumper 116 can be composed of other flexible or rigid materials, such as plastic or metal. The distal bumper 116 can be hollow, air-filled, or fluid-filled. While the distal bumper 116 is preferably attached to the surface of the tubing unit 102 with adhesive, the distal bumper 116 can alternatively be clasped, sutured, wrapped around, or tied to the tubing unit 102. In the preferred embodiment, the distal bumper 116 is integral to the tubing unit 102 such that it is fixed to the tubing unit 102 in order to keep the tubing unit 102 in the correct position on the patient's body. In a further alternative, the distal bumper 116 can be manufactured as an integral component of the tubing unit 102. In one example where the tubing unit 102 is formed through extrusion, an extending ridge that serves as the distal bumper 116 can be directly formed during this extrusion process. Preferably, the distal bumper 116 fully circumscribes 154 the tubing unit 102 (as shown in FIG. 7A). Alternatively, as shown in FIG. 7B, the distal bumper 116 partially circumscribes 156 the tubing unit 102. In yet another embodiment, as shown in FIG. 7C, the distal bumper 116 consists of any number of appendages 158 that extend outwardly from the surface of the tubing unit 102.

The proximal bumper 118 is configured to be located on the tubing unit 102 at the most distal location of the portion of the tubing unit 102 that is located outside the body once the feeding tube assembly 100 is placed. That is, the proximal bumper 118 is located on the tubing unit 102 just outside of the abdominal wall 120. While the proximal bumper 118 may be manufactured integral to the tubing unit 102, the proximal bumper 118 is preferably affixed to the tubing unit 102 after placement of the tubing unit 102 in the patient's body. The proximal bumper 118 functions to prevent migration of the feeding tube assembly 100 and is specifically configured to prevent the proximal end 104 of the tubing unit 102 from moving distally past the exterior abdominal wall 120 and thereby entering the body. In the preferred embodiment, the proximal bumper 118 extends 1-5 mm outwardly from the outer surface of the tubing unit 102. Alternatively, any dimension that prevents the proximal end 104 from entering the body is appropriate.

Various embodiments of the proximal bumper 118 are shown in FIG. 7. In the preferred embodiment, the proximal bumper 118 is a ring composed of the same flexible material as the tubing unit 102, so that it minimizes the chance of obstruction or harm to the patient as it is being placed. Alternatively, the proximal bumper 118 can be composed of plastic, metal, or other solid rigid or flexible materials. Alternatively, the proximal bumper 118 can be hollow, air-filled, or fluid-filled. The proximal bumper 118 is preferably held in place on the surface of the tubing unit 102 frictionally. In alternative embodiments, however, the proximal bumper 118 can be clasped, sutured, wrapped around, or tied to the tubing unit 102, or may even be affixed using an adhesive material. In some embodiments, the proximal bumper 118 may be adjustably slidable along the length of the tubing unit 102 to allow for patient customization as well as to ensure the most secure fit. In a further alternative, the proximal bumper 118 can be manufactured directly into the tubing unit 102. In one example where the tubing unit 102 is formed through extrusion, an extending ridge that serves as the proximal bumper 118 can be directly formed during this extrusion process. Preferably, the proximal bumper 118 fully circumscribes 154 the tubing unit 102. Alternatively, the proximal bumper 118 can partially circumscribe 156 the tubing unit 102, or consist of any number of appendages 158 that extend outwardly from the surface of the tubing unit 102.

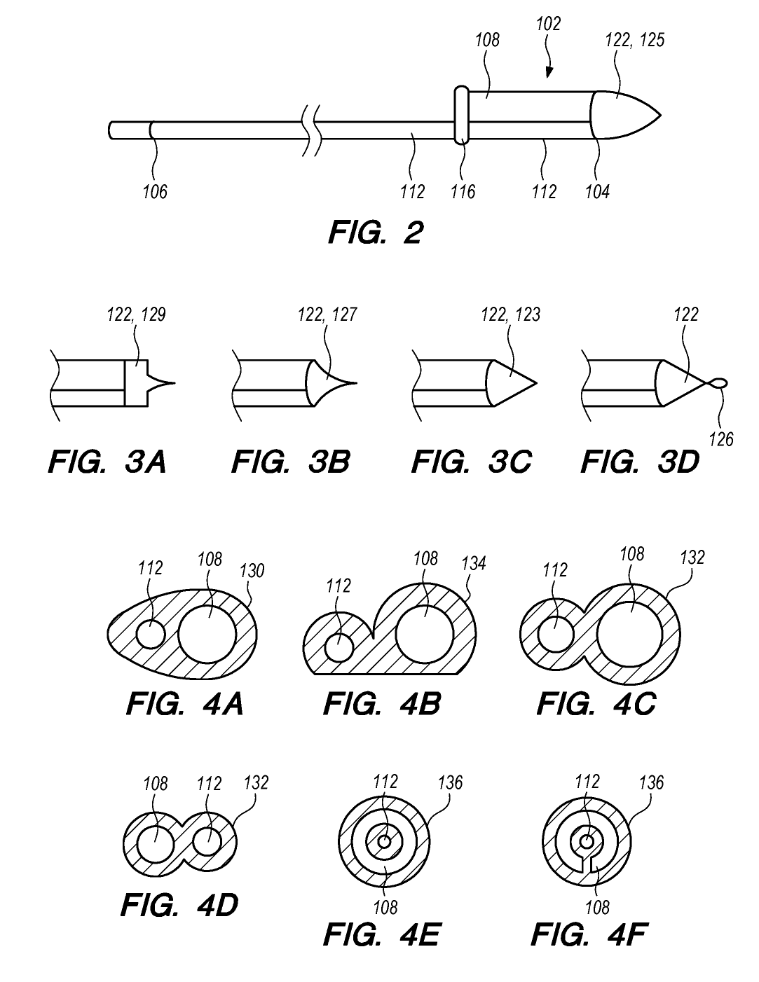

The pointed tip attachment piece 122 functions to bluntly dissect the gastric lining 113 as the tubing unit 102 is endoscopically pulled out of the body through the abdominal wall 120. In the preferred embodiment, the pointed tip attachment piece 122 is conical in shape 123 and composed of a rigid plastic configured to bluntly dissect through the abdominal wall 120, as shown in FIG. 3C. Alternatively, the pointed tip attachment piece 122 can be made of metal. The pointed tip attachment piece 122 is preferably attached to the proximal end 104 of the tubing unit 102 with adhesive. In alternative embodiments, the pointed tip attachment piece 122 can have profiles of varying degrees of curvature 125 (as shown in FIG. 2), 127 (as shown in FIG. 3B), or any other geometry that allows the feeding tube assembly 100 to be capable of dissection, as shown in FIGS. 2-3D. In one example, the pointed tip attachment piece 122 can have a sharp needle-like tip 129 (FIG. 3A). In other embodiments, the pointed tip attachment piece can be manufactured into the tubing unit 102, press fit into the proximal end of the tubing unit 102, secured with a barbed fit within a single or both lumens 108, 112, clamped around the outer surface of the tubing unit 102, or otherwise attached to the tubing unit 102. In the preferred embodiment, the length of the pointed tip attachment piece 122 is less than 2 cm. Once the pointed tip attachment piece 122 has bluntly dissected the abdominal wall 120 and is located outside the body, it is preferably cut off from the tubing unit 102. Alternatively, the pointed tip attachment piece 122 can snap, break, or twist off from the tubing unit 102 once the feeding tube assembly 100 has been placed.

In the preferred embodiment, the pointed tip attachment piece 122 further comprises a wire loop 126 (also referred to as appendage piece) extending from its tip (as shown in FIG. 3D) that is configured to be attachable to the pull-through wire that attaches to the pointed tip attachment piece 122 and pulls it down the esophagus and through the abdominal wall 120 (as described below). In alternative embodiments, this appendage piece 126 can be a thread, a suture, or a hook. In other alternative embodiments, the appendage piece 126 can be a slit, hook, hole, loop, or other feature that is directly manufactured within or onto the pointed tip attachment piece 122. In other alternatives, a magnetic, adhesive, or otherwise attractive component within or on the pointed tip attachment piece 122 can serve to attach it to the pull-through wire.

In some embodiments, as shown in FIG. 10, the proximal end 104 of the tubing unit 102 can be split between the lumens 108, 112 in such a way that both lumens 108, 112 are preserved and remain separate from each other, thereby forming two tubes 108, 112 connected at their distal ends 104. In one embodiment, the tubing unit 102 can be manufactured to be perforated or otherwise peel-able, such that the tubing unit 102 can be peeled apart into two tube pieces 108, 112 connected at their distal ends. Alternatively, a scalpel may be used to make a longitudinal cut between the lumens 108, 112 after the feeding tube assembly 100 has been placed and secured within the patient. As indicated previously, a retention band 124 on the tubing unit prohibits splitting of the tubing beyond the point desired. By splitting the lumens 108, 112 at the proximal end 104 of tubing unit 102, two separate heads 142 can be attached to the tubing unit 102—one secured within the gastric lumen 108 and one within the jejunal lumen 112—instead of having a single head with two ports attached to the proximal end 104 of the joined tubing unit 102. This can serve to reduce the bulkiness of the segment of the tubing unit 102 that remains outside of the body, which may be more comfortable or aesthetically appealing to the patient. Furthermore, increasing the distance between the heads 142 may facilitate easier attachment of the various external components required for feeding, such as syringes that deliver medicine and nutrients. For example, by having separate heads 142, there may be enough room to have a syringe delivering nutrients attached to the jejunal head while a syringe administering medicine is simultaneously attached to the gastric head.

If the proximal end 104 of the tubing unit 102 is split, the feeding tube assembly 100 may further comprise a single or series of retention bands 124, which function to prevent the tubing unit 102 from further splitting distally. The retention band(s) 124 is located on the portion of the tubing unit 102 external the patient's body and is located toward the proximal end of the tubing unit. A medical professional may cut the tubing unit to a desired length to remove unnecessary tubing. This retention band 124 prohibits the tubing unit 102 from further splitting beyond what is purposefully done by the medical professional, thus keeping the tubing unit 102 in one integrated assembly. Various embodiments of the retention band are shown in FIGS. 6A-6D. Preferably, the retention band 124 is an elastic band 144 that tightly encircles the tubing unit 102 (as shown in FIG. 6A). Adhesives or other means of securing the band 124 to the surface of the tubing unit 102 can be used to ensure that the band 124 does not slip off of the tubing unit 102. Alternatively, the retention bands 124 can be composed of more rigid materials, such as metals, that fully or partially encircle the tubing unit 102. In another alternative, the retention band 124 can be a tie, suture, or other material that wraps 146 around the tubing unit 102 (as shown in FIG. 6B). Furthermore, a clamp 148 (FIG. 6C) or other means for pressing opposing sides of the tubing unit 102 together may be used. Furthermore, circumferential ridges 150 of the tubing material can be manufactured onto the tubing unit's 102 surface and serve as the retention band 124 (as shown in FIG. 6A). If perforation is used for the splitting, the perforation can be manufactured only along the length that is desired to be split. In another alternative, a change in method of manufacture between the split region and the connected region can be used to serve as a retention band 124. For example, the tubing unit 102 distal to the split region can be manufactured as a single extruded piece, and the split region extruded as separate tubes. The separate tubes can then, for instance, be adhered together with adhesive to keep them joined while the tube is being placed; after the tube is placed, they can be peeled apart. In this example, the change in extrusion from single piece to separate tubes mechanically demarcates these regions, thereby serving as the retention band 124. In a further alternative, the retention band 124 can perform the splitting. For example, in one embodiment, the retention band 124 can have a sharp center piece 152 that separates the tube as the band is moved distally along the tubing unit 102 (as shown in FIG. 6D).

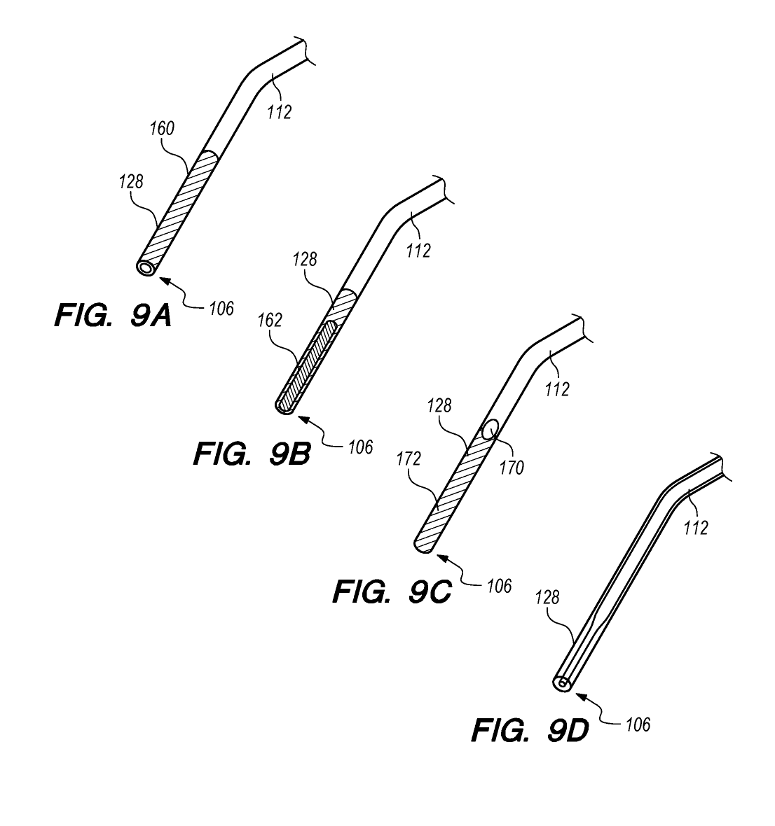

As shown in FIG. 1, the feeding tube assembly 100 can further comprise a weighted tip attachment piece 128 located at the distal end of the jejunal lumen 112, 106. The weighted tip 128 functions to decrease the time required for the distal end 106 of the tubing unit 102 to migrate into its final location as well as to prevent significant proximal migration of the jejunal lumen 112 once placed, with the weighted tip 128 having a greater density than that of the tubing unit 102. In the preferred embodiment, the weighted tip 128 is made of a rigid plastic. In other embodiments, the weighted tip 128 is made of a metal, flexible rubber, or any other material with a density satisfying the requirement described. FIGS. 8A-9D show alternate embodiments of the weighted tip 128. In the preferred embodiment (FIG. 9A), the weighted tip 128 is constructed as a hollow cylinder 160 that is adhered to the most distal portion 106 of the tubing unit 102, allowing materials to flow through the jejunal lumen 112 without increased obstruction. In another embodiment, the weighted tip 128 is a hollow cylinder 162 with an outer surface that is confluent with the outer surface of the jejunal lumen 112 with the opening in the weighted tip extending down the side of the weighted tip 162 (FIG. 9B). In further alternative embodiments, as shown in FIGS. 8A-8B, the weighted tip 128 consists of a single or series of appendages. These appendages can hang or extend from the distal end 106 and be attached in various ways within the jejunal lumen 112 or on the surface of the distal end 106 of the tubing unit 102. For example, the weighted tip 128 can consist of a rigid plastic piece 164 that is attached by a wire secured inside the tubing unit 102. In a second example, a rigid plastic piece or multiple pieces 166 can be attached by a wire secured to the surface of the most distal portion 106 of jejunal lumen 122 of tubing unit 102. In yet another embodiment, as shown in FIG. 9C, the weighted tip 128 closes off the jejunal outlet, in which case the tubing unit 102 would have an outlet or multiple outlets 170 located on its side. For example, the weighted tip 128 could be a solid cylinder 172 that is attached to the inside of the jejunal lumen 112 and fully or partially occludes flow from this outlet; the fluids would then pass through an outlet 170 located proximally on the side of the tube. Finally, one embodiment of the weighted tip128 (FIG. 9D) is a seamless continuation of the jejunal tube 112 but the distal end 106 has a smaller average inner diameter (i.e. the opening of the tube is smaller, therefore the amount of tubing at the distal end 106 increases) than the remaining portion of jejunal tube 112 to achieve an overall higher density.

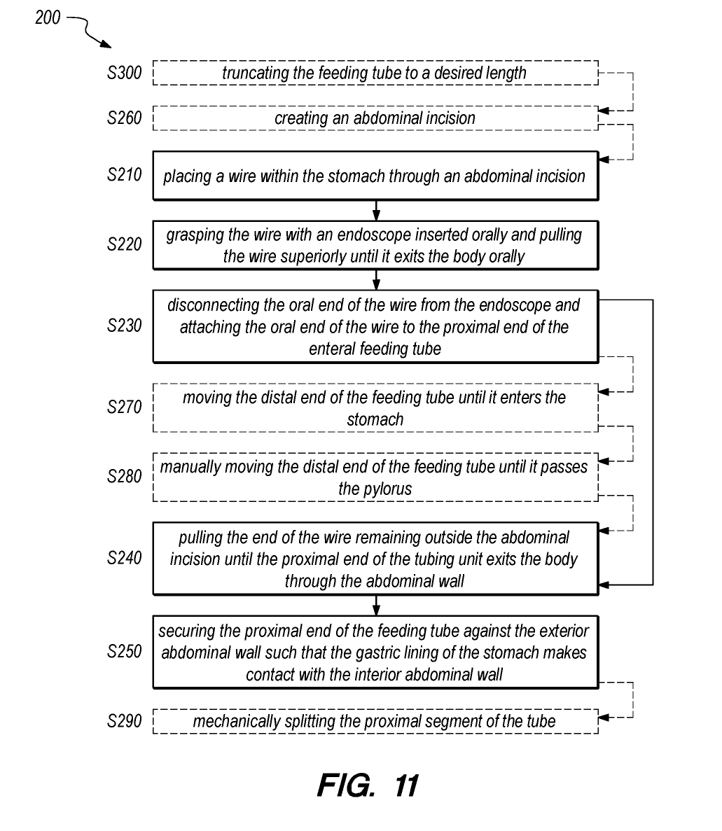

Turning now to FIG. 11, a method for use of the feeding tube assembly 100 of the present invention may be described. Generally, the feeding tube assembly 100 of the present invention is placed in a patient such that the proximal end 104 of the tubing unit 102 is exposed outside of the patient's abdominal wall 120 (as shown in FIG. 1) while a portion of the tubing unit 102 (the portion being characterized as the most distal end of the dual-lumen length, defined above) is inside the patient's body. Because the gastric lumen 108 and jejunal lumen 112 are joined together to create the tubing unit 102, the tubes 108, 112 can be placed simultaneously in a single procedure rather than in multiple independent procedures. Generally speaking, the method is preferably done endoscopically using the “pull” method. That is, the feeding tube assembly is introduced orally and pulled through the patient's body until the proximal end of the tubing unit extends through an incision in the abdomen wall. This method of placing the feeding tube assembly 100 into the patient's body is described more fully below.

As shown the method 200 for endoscopically placing a gastro-jejunal enteral feeding tube assembly 100 preferably includes: placing a pull-through wire in the patient's stomach 110 through an abdominal incision in the abdominal wall 120 at step S210; grasping the pull-through wire with an endoscope inserted orally and pulling the pull-through wire superiorly until one end of the pull-through wire exits the body orally at step S220 (while the other end of the pull-through wire remains exposed outside of the abdominal incision); disconnecting the oral end of the pull-through wire from the endoscope and attaching the oral end of the pull-through wire to the proximal end 104 of the tubing unit 102 of the enteral feeding tube assembly 100 at step S230; pulling the abdominal end of the pull-through wire (which remains outside the abdominal incision) until the proximal end 104 of the tubing unit 102 exits the body through the abdominal wall 120 at step S240; and securing the proximal end 104 of the tubing unit 102 of the feeding tube assembly 100 against the exterior abdominal wall 120 such that the gastric lining 113 of the stomach 110 makes contact with the interior abdominal wall at step S250. The method 200 can further comprise: creating an abdominal incision at step S260; moving the distal end 106 of jejunal lumen 112 of the feeding tube assembly 100 until it enters the stomach 110 at step S270; moving the distal end 106 of the jejunal lumen 112 of the feeding tube assembly 100 until it passes the pylorus 111 at step S280; mechanically splitting the proximal segment of the tubing unit 102 located outside the body at step S290; and truncating the feeding tube assembly 100 to a desired length at step S300.

Block S210 recites the step of placing a pull-through wire into the stomach 110 through an abdominal incision such that one end of the pull-through wire is located inside the patient's body and the other end remains outside of the patient's body through the abdominal incision. Step S210 functions to begin the endoscopic pull-through method in which the pull-through wire pulls the feeding tube assembly 100 from the oral cavity and out the abdominal wall 120. Step S210 is preferably implemented with a single metal pull-through wire that is capable of puncturing through the gastric lining 113. In the preferred method, at step S210 the surgeon places one end of the pull-through wire through the abdominal incision and pushes the pull-through wire until it punctures through the gastric lining 113 and enters the stomach 110. Preferably, step S210 is performed without medical imaging technology, such as internal videography or fluoroscopy. Alternatively, for instance, a video endoscope can also be placed through the abdominal incision to ensure that the pull-through wire appropriately punctures the gastric lining 113.

Block S220 recites the step of grasping the pull-through wire with an endoscope inserted orally and pulling the pull-through wire superiorly until one end exits the body orally. Step S220 functions to provide a means for the feeding tube assembly 110 to enter the body orally, and is preferably implemented with the pull-through wire used in step S210 and an endoscope having imaging capabilities and configured to attach to the pull-through wire. In the preferred embodiment, the endoscope is inserted orally and is pushed inferiorly through the esophagus until it reaches the stomach 110. Using the imaging capabilities of the endoscope, the surgeon positions the tip of the endoscope near the end of the pull-through wire. The endoscope preferably attaches to the pull-through wire by grabbing the end of the pull-through wire with forceps located at the endoscope tip. Alternatively, the endoscope may be configured with a looped end, hook, or other appendage that otherwise secures the pull-through wire to the endoscope tip. In further alternative embodiments, the endoscope wraps or ties the pull-through wire around itself. Mechanical or adhesive components of the endoscope can alternatively be utilized to attach to the pull-through wire. Upon attachment of the endoscope and the pull-through wire, the endoscope is pulled superiorly until the endoscope tip and one end of the pull-through wire exit the body orally. Step S220 may further comprise holding onto or otherwise securing the opposite end of the pull-through wire outside the abdominal wall 120 to prevent it from entering the body if the oral end is pulled too far.

Block S230 recites the step of disconnecting the oral end of the pull-through wire from the endoscope and attaching the oral end of the pull-through wire to the proximal end 104 of the enteral feeding tube assembly 100. Step S230 functions to secure the feeding tube assembly 110 to the pull-through wire so that it can be pulled through the body in the direction opposite to step S220. Step S230 is preferably implemented with the pull-through wire and endoscope mentioned previously, and the feeding tube assembly 100 preferably includes a proximal end 104 configured for blunt dissection, as described more fully above. Alternatively, any other feeding tube assembly with two lumens mechanically constrained to move together may be implemented in this step. In the preferred method, the oral end of the pull-through wire is detached from the endoscope by releasing the forceps, clamp, or other mechanism that was holding it. Alternatively, the pull-through wire can be untied from the endoscope, unwrapped, pulled off, cut off, or otherwise removed. The pull-through wire is preferably looped through the pointed tip attachment piece 122 of the feeding tube assembly's 100 proximal end 104 and securely tied to the feeding tube assembly 100. Alternatively, the wire can be wrapped or tied around the pointed tip attachment piece 122 or around the tubing unit 102. In further alternative embodiments, the wire can be sutured into or magnetically or adhesively attached to the feeding tube assembly 100. As described above, in one embodiment the feeding tube assembly 100 may comprise an appendage 126 attached to the pointed tip attachment piece 122. This appendage 126 may be utilized in this step, where the end of the oral wire is attached to the appendage 126, thereby allowing the feeding tube assembly 100 to be pulled through the body, as described below.

Block S240 recites the step of pulling the abdominal end of the pull-through wire remaining outside the abdominal incision until the proximal end 104 of the tubing unit 102 exits the body through the abdominal wall 120. As described above, the pull-through wire was previously inserted through the incision and into the patient's body and pulled upwardly through the esophagus and out of the patient's mouth. In this step, the pull-through wire (with feeding tube attached) is pulled in the opposite direction such that the feeding tube assembly is inserted orally and travels through the esophagus and stomach until the proximal end 104 of the tubing unit 102 extends through the incision in the abdominal wall 120. Step S240 functions to bring the proximal end 104 of the feeding tube assembly 100 outside the body, so that nutrients can be administered externally. Step S240 is preferably implemented with the pull-through wire described in previous blocks and the feeding tube assembly 100 described herein; however, any gastro-jejunal feeding tube with a proximal end configured for blunt dissection can be used. In the preferred method, the doctor grasps the end of the abdominal end of the pull-through wire with his hand and pulls anteriorly away from the abdomen wall 120 until the proximal end 104 of the tubing unit 102 exits the body. In alternative embodiments, the pull-through wire can be grasped with forceps, or otherwise attached to an object that then pulls the pull-through wire away from the abdominal wall 120.

Block S250 recites the step of securing the proximal end 104 of the feeding tube assembly 100 against the exterior abdominal wall 120 such that the gastric lining 113 of the stomach 110 makes contact with the interior abdominal wall 120. Step S250 functions to secure the positioning of the feeding tube assembly 110 so that the proximal end 104 does not migrate into the body and the distal end 106 does not exit the body. Step S250 is preferably implemented with the feeding tube assembly 100 of the present invention, but is not so limited. In the preferred embodiment, the user grasps the exposed proximal end 104 of the tubing unit 102 and pulls it anteriorly away from the abdomen 120 until a distal bumper 116 on a segment of the feeding tube unit 102 within the gastric lining 113 pulls the gastric lining 113 into contact with the interior abdominal wall 120. If a tubing unit 102 without a distal bumper 116 is used, an alternative metric, such as the length of exposed tubing outside the abdomen, can be used to determine when the proximal end 104 of the tubing unit 102 has been pulled by a sufficient amount. Once the tubing unit 102 has been positioned, a proximal bumper 118 is preferably attached to the tubing unit 102 at the distal end of the exposed length such that it is in contact with the exterior abdominal wall 120. In the preferred embodiment, the proximal bumper 118 fully circumscribes the tubing unit 102 and is placed around the proximal end 104 and slid distally until it is secured at its desired location adjacent the abdominal wall 120. Alternatively, the bumper 118 can be manufactured on the surface of the tubing unit 120 during the time of production, tied around the tubing unit 120, clamped around the tubing unit 102, or secured in any other manner such that the bumper 118 is configured to prevent the proximal end 104 of the feeding tube assembly 100 from entering the abdomen. In embodiments where the tubing unit 102 does not have a proximal bumper 118, the tubing unit 102 can be adhered to the abdominal wall 120 adhesively or in any other way that maintains the desired length or range of lengths of tubing outside the body.

Method 200 can further comprise step S260, which would precede step S210 and include creating an abdominal incision. Step S260 functions to create an entry point for the wire described in step S210 to enter the abdominal cavity. This is preferably done by cutting through the abdominal wall 120 with a scalpel. Alternatively, a biopsy punch, surgical scissors, or other tool configured to penetrate through the abdominal wall 120 can be used. Step S210 may further comprise any combination of the following: sterilizing the abdomen prior to the incision, sterilizing the equipment prior to the incision, and drawing the desired incision path on the patient's abdomen.

Method 200 can further comprise step S270, which involves moving the distal end 106 of the feeding tube assembly 100 until it exits the esophagus. Step S270 functions to clear the esophagus, which helps prevent obstruction and occlusion of this passageway. Step S270 can occur at any point in method 200 after step S230, and is preferably implemented with an endoscope. In the preferred embodiment, the endoscope pushes the distal end 106 of the feeding tube assembly 100 inferiorly until it exits the esophagus. Alternatively, the endoscope can be attached to the distal end 106 or other portion of the feeding tube assembly 100 and then dragged, pushed, or pulled inferiorly until the feeding tube assembly 100 exits the body. In alternative embodiments, the distal end 106 of the feeding tube assembly 100 can be moved by flushing the esophagus with water or another fluid, causing a pressure difference whereby the tube is forced down the esophagus, or other alternative techniques that function to move the feeding tube 100 inferiorly. As noted above, the distal end 106 may further comprise a weighted tip 128 that aids in clearing the distal end 106 from the patient's esophagus. The weighted tip 128 assists the distal end 106 in migrating into the patient's jejunum and reduces the risk of the distal end migrating back into the patient's esophagus. In one alternative method of use of the feeding tube assembly 100, radiopaque or fluorescent markers are blended with the jejunal lumen 112 material so that the jejunal lumen 112 can be monitored radioactively or fluorescently in order to track the migration process of the jejunal lumen 112.

Step S270 can further comprise step S280, which includes manually moving the distal end 106 of the feeding tube assembly 100 until it passes the pylorus 111. Step S280 functions to directly place the jejunal lumen 112 of the feeding tube assembly 100 into the jejunum 114 instead of waiting for it to drift there by natural peristalsis. In the preferred embodiment, step S280 is performed immediately following step S270. Alternatively, step S280 can be performed any time after the distal end 106 of the feeding tube assembly 100 has entered the stomach 110. In the preferred embodiment, an endoscope is used to push the distal end 106 of the feeding tube assembly 100 inferiorly until it passes the pylorus 111 or reaches a desired depth within the jejunum 114. Alternatively, the endoscope can be attached to the distal end 106 or other portion of the jejunal lumen 112 and drag, push, or pull it inferiorly until this desired location is reached.

Method 200 can further comprise step S290, which recites mechanically splitting the proximal segment of the tubing unit 102 located outside the body (the dual-lumen length of tubing unit 102). This is preferably implemented with the feeding tube assembly 100 of the present invention, but applies to any feeding tube that is a single piece where the gastric and jejunal lumens are constrained to move together. Step S290 functions to form two separate tubes, one gastric 108 and the other jejunal 112, along the proximal length of the tubing unit 102 that is located outside the body. This increases the distance between the inlets and allows them to have separate heads, which can ease the administration of nutrients and aspiration of gastric contents. In the preferred embodiment, a scalpel is used to cut the tubing unit 102 longitudinally between the lumens 108, 112 starting at the proximal end 104 of the tubing unit 102. Alternatively, the tubing unit 102 can otherwise be cut or peeled apart to form the separate tubes 108, 112. Step S290 can further comprise placing, adhering, or fixing a retention band 124 to the tubing unit's 102 outer surface at the distal end of the split section to prevent further splitting. In the event a sheath envelops the proximal end 104 of the tubing unit 102, the method further comprises the step of peeling the sheath off the tubing unit from the proximal end 104 to expose the jejunal tube 112 and gastric tube 108. This peeling may be in a direction parallel to the longitudinal axis of the tubes, in a direction perpendicular to the axis, or in a spiral direction around the tubes.

Method 200 can further comprise step S300, which recites truncating the feeding tube assembly 100 to a desired length. This functions to allow the feeding tube assembly 100 to be customizable to the size and anatomy of a wide range of different patients. Step S300 is preferably performed prior to step S200. In the preferred embodiment, a length of the feeding tube assembly 100 at either the distal 106 or proximal end 104 is cut off with a scalpel. Alternatively, the tubing unit 102 can be cut with scissors or any other tool configured to cut through the tubing material. Alternatively, the tubing unit 102 can be marked for cutting at another stage in method 200. In alternative embodiments, the tube can be truncated at any other point during or after method 200. Finally, after successful treatment of the patient, it may be desirable to remove the feeding tube assembly 100 from the patient's body. One method for removing the feeding tube assembly 100 include pulling the assembly 100 directly out of the patient's body by pulling the proximal end 104 of the tubing unit 102 away from the abdominal wall 120 until the entire assembly 100 is removed.

The present invention has been described with reference to certain preferred and alternative embodiments that are intended to be exemplary only and not limiting to the full scope of the present invention as set forth in the appended claims

1. A dual-lumen enteral feeding assembly for placement in a patient's body comprising:
a. a gastric lumen having a first end, a second end, and a length of lumen between the first end and second end;
b. a jejunal lumen having a first end, a second end, and a length of lumen between the first end and second end;
c. a pointed tip attachment piece having an open first end and a pointed second end, wherein the first end of the pointed tip attachment piece is adjacent to the second end of the jejunal lumen and the second end of the gastric lumen;
d. an interior bumper adjacent to the jejunal lumen and gastric lumen, wherein the interior bumper is located at the first end of the gastric lumen; and
e. a weighted tip located at the first end of the jejunal lumen.

2. The dual-lumen enteral feeding assembly of claim 1, further comprising an exterior bumper adjacent to the jejunal lumen and gastric lumen.

3. The dual-lumen enteral feeding assembly of claim 1, comprising at least one band circumscribing the jejunal lumen and gastric lumen.

4. The dual-lumen enteral feeding assembly of claim 1, further comprising an appendage connected to the second end of the pointed tip attachment piece and configured to receive a pull-through wire.

5. A dual-tube enteral feeding assembly for placement in a patient's body comprising:
a. a gastric tube having a first end, a second end, and a length of tube between the first end and second end;
b. a jejunal tube having a first end, a second end, and a length of tube between the first end and second end,;
c. a pointed tip attachment piece having an open first end and a pointed second end, wherein the first end of the pointed tip attachment piece is adjacent to the second end of the jejunal tube and the second end of the gastric tube;
d. an interior bumper adjacent to the jejunal tube and gastric tube, wherein the interior bumper is located at the first end of the gastric tube; and
e. a weighted tip located at the first end of the jejunal tube, wherein the weighted tip is configured to migrate the first end of the jejunal tube into a jejunum of the patient.

6. The dual-tube enteral feeding assembly of claim 1, further comprising an exterior bumper adjacent to the jejunal tube and gastric tube.

7. The dual-tube enteral feeding assembly of claim 1, comprising at least one band circumscribing the jejunal tube and gastric tube.

8. The dual-tube enteral feeding assembly of claim 1, further comprising an appendage connected to the second end of the pointed tip attachment piece and configured to receive a pull-through wire.

9. A method for placing an enteral feeding tube assembly into a patient's body, comprising the steps of:
a. abdominally inserting a first end of a pull-through wire into a stomach of the patient through an abdominal incision in the patient;
b. orally inserting a first end of an endoscope into the patient's stomach;
c. grasping the first end of the pull-through wire with the first end of the endoscope;
d. orally removing the first end of the endoscope from the patient's body, thereby orally removing the first end of the pull-through wire from the patient's body;
e. disconnecting the first end of the pull-through wire from the first end of the endoscope;
f. connecting a first end of the feeding tube assembly to the first end of the pull-through wire;
g. pulling a second end of the pull-through wire through the abdominal incision until the first end of the pull-through wire and the first end of the feeding tube assembly abdominally exit the patient's body through the abdominal incision; and
h. securing the feeding tube assembly such that a gastric lining of the patient's stomach makes contact with an interior abdominal wall of the patient, wherein the first end of the feeding tube assembly is outside the patient's body and a second end of the feeding tube assembly is inside the patient's body.

10. The method of claim 9, further comprising the step of splitting the first end of the feeding tube assembly into a gastric tube and a jejunal tube.

11. The method of claim 9, further comprising the step of reducing a length of the feeding tube assembly by cutting at least one of the first end and second end of the feeding tube assembly.

12. The method of claim 9, wherein the securing the feeding tube assembly step comprises pulling the first end of the feeding tube assembly until an interior bumper located between the first end and second end of the feeding tube assembly causes the gastric lining of the patient to make contact with the interior abdominal wall of the patient, whereby the interior bumper prevents the second end of the feeding tube assembly from exiting the patient's body.

13. The method of claim 9, further comprising the step of affixing an exterior bumper to the feeding tube assembly such that the exterior bumper is in contact with the outer abdominal wall of the patient, thereby preventing the first end of the feeding tube assembly from moving into the patient's body.
